(19) United States
(12) Patent Application Publication
(10) Pub. No.: US 2011/0059501 A1
(43) Pub. Date: Mar. 10, 2011

(54) Protein Glycosylation

(75) Inventor: Benjamin Guy Davis, Oxford (GB)

(73) Assignee: ISIS INNOVATION LIMITED, Oxford (GB)

(21) Appl. No.: 11/910,883

(22) PCT Filed: Apr. 6, 2006
(86) PCT No.: PCT/GB06/01274

§ 371 Date: Nov. 29, 2010

(30) Foreign Application Priority Data
Apr. 8, 2005 (GB) 0507123.8

(51) Int. Cl. C12N 9/00; C07K 14/00; C07K 7/06; C07K 7/08; C07K 14/575; C07K 14/54; C07K 16/00; C07K 14/505; C07K 14/555

(52) U.S. Cl. 435/183; 530/395; 530/328; 530/327; 530/326; 530/325; 530/324; 530/380; 530/399; 530/351; 530/387.1; 530/397

(57) ABSTRACT

The present invention relates to methods for glycosylating a protein in which the protein is modified to include an alkyne and/or an azide group. The invention further relates to a protein glycosylated by these methods.

### FIELD OF THE INVENTION

The present application is concerned with methods for the glycosylation of proteins and the glycosylated proteins provided by these methods.

### BACKGROUND TO THE INVENTION

The co- and post-translational glycosylation of proteins plays a vital role in their biological behaviour and stability (R. Dwek, Chem. Rev., 96:683-720 (1996)). For example, glycosylation plays a major role in essential biological processes such as cell signalling and regulation, development and immunity. The study of these events is made difficult by the fact that glycoproteins occur naturally as mixtures of so-called glycoforms that possess the same peptide backbone but differ in both the nature and the site of glycosylation. Furthermore, since protein glycosylation is not under direct genetic control, the expression of therapeutic glycoproteins in mammalian cell culture leads to heterogeneous mixtures of glycoforms. The ability to synthesise homogeneous glycoprotein glycoforms is therefore not only a prerequisite for accurate investigation purposes, but is of increasing importance when preparing therapeutic glycoproteins, which are currently marketed as multi-glycoform mixtures (e.g. erythropoietin and interleukins). Controlling the degree and nature of glycosylation of a protein therefore allows the possibility of investigating and controlling its behaviour in biological systems.

A number of methods for the glycosylation of proteins are known, including chemical synthesis. Chemical synthesis of glycoproteins offers certain advantages, not least the possibility of access to pure glycoprotein glycoforms. One known synthetic method utilises thiol-selective carbohydrate reagents, glycosylmethane thiosulfonate reagents (glyco-MTS). Such glycosylmethane thiosulfonate reagents react with thiol groups in a protein to introduce a glycosyl residue linked to the protein via a disulfide bond (see for example WO00/01712).

Cu(I) catalyzed triazole formation has been used for a number of labelling studies (Link et al., J. Am. Chem. Soc. 125: 11164-11165 2003; Link et al., J. Am. Chem. Soc. 126: 10598-10602 2004; and Speers et al., Chemistry and Biology 11: 535-546 2004) as well as in synthesis (Tomoe et al., J. Org. Chem. 67(9): 3057-3064 2002). The attractive features of this reaction are the high selectivity of the reaction of azides with alkynes and the ability to perform the reaction under aqueous conditions in the presence of a variety of other functional groups.

In the recent literature (Kuijpers et al., Org. Lett. 6(18):3123-3126 2004) synthesis of triazole-linked glycosyl amino acids and small glycopeptides from suitably functionalised protected carbohydrates and protected amino acids/peptides has been demonstrated. Also other types of triazole linked glycoconjugates were reported (Chittaboina et al., Tetrahedron Lett. 46:2331-2336 2005) which were synthesized utilizing protected carbohydrate derivatives.

Lin and Walsh modified a 10 amino acid cyclic peptide, N-acetyl cysteamine thoiester (SNAC) to introduce alkyne functionality into the peptide. The method involved substituting amino acids in the peptide with the unnatural amino acid analogue, propargylglycine, at different positions in the peptide (Van Hest et al. J. Am. Chem. Soc. 122:1282-1288 (2000) and Kiick et al., Tetrahedron 56:9487-9493 (2000)). The modified peptides were then conjugated to azido sugars to produce glycosylated cyclic peptides.

There is a need for a simplified method, for example one which does not require the use of protected glycosylating reagents, for glycosylation of more complex structures, for example proteins, than described in the prior art and which allows glycosylation at multiple sites in a wide range of different proteins.

### STATEMENTS OF THE INVENTION

According to a first aspect of the present invention there is provided a method for modifying a protein, the method comprising modifying the protein to include at least an alkyne and/or an azide group.

As used herein an “azide” group refers to (N═N═N) and an “alkyne” group refers to a CC triple bond.

The modification to the protein generally involves the substitution of one or more amino acids in the protein with one or more amino acid analogues comprising an alkyne and/or azide group. Alternatively, or in addition to the foregoing, the modification to the protein may include the introduction of one or more natural amino acids into the protein as discussed herein. In another alternative, the modification to the protein may involve the modification of a side chain of an amino acid to include a chemical group, for example a thiol group. The modification of the protein to include an azide, alkyne or thiol group typically occurs at a pre-determined position within the amino acid sequence of the protein.

In a preferred aspect of the invention the modification to the protein involves the substitution of one or more amino acids in the protein with one or more non-natural (ie. non-naturally occurring) amino acid analogues. The non-natural amino acid analogue may be a methionine analogue. The methionine analogue may be homopropargyl glycine (Hpg) (Van Hest et al., J. Am. Chem. Soc., 122, 1282-1288 (2000)), homoallyl glycine (Hag) (Van Hest et al., FEBS Letters, 428, 68-70 (1998)) and/or azidohomoalanine (Aha) (Kiick et al., Proc. Natl. Acad. Sci. USA, 99, 19-24 (2002), preferably homopropargyl glycine.

The modification of the protein to introduce one or more unnatural amino acids, for example methionine analogues, may be achieved by methods known in the art, see for example Van Hest et al., J. Am. Chem. Soc. 122, 1282-1288 (2000). Specifically the modification of a protein to introduce one or more methionine analogues involves the site directed mutagenesis to insert into a nucleic acid sequence encoding the protein the codon AUG coding for methionine. Preferably the insertion of the codon for methionine occurs at a pre-determined position within the nucleic acid sequence encoding the protein, for example at a location within a region of the nucleic acid sequence that encodes the N-terminus (or amino end) of the protein. Expression of the protein can then be achieved by translating the nucleic acid sequence containing the inserted methionine codon in an auxotrophic methionine-deficient bacterial strain in the presence of methionine analogues, for example, Aha or Hpg.

The method of the invention may involve the modification of the protein to include an alkyne group by the step of substituting one or more amino acids in the protein with homopropargyl glycine or homoallyl glycine.

Alternatively, or additionally, the method invention may involve the modification of the protein to include an azide group by the step of substituting one or more amino acids in the protein with azidohomoalanine.

Preferably the method of the invention involves the modification of the protein to include an azide group (as described herein) and an alkyne group (as described herein).

The term “protein” in this text means, in general terms, a plurality (minimum of 2 amino acids) of amino acid residues (generally greater than 10) joined together by peptide bonds. Any amino acid comprised in the protein is preferably an a amino acid. Any amino acid may be in the D- or L-form.

In a preferred aspect of the invention the protein comprises a thiol (—SH) group for example present in one or more cysteine residues. The cysteine residue(s) may be naturally present in the protein. Where the protein does not include a cysteine residue, the protein may be modified to include one or more cysteine residues. A thiol group(s) may be introduced into the protein by chemical modification of the protein, for example to introduce a thiol group into the side chain of an amino acid or to introduce one or more cysteine residues. Alternatively a thiol containing protein may be prepared via site-directed mutagenesis to introduce a cysteine residue. Site directed mutageneis is a known technique in the art (see for example WO00/01712). Specifically, a cysteine residue may be introduced into the protein by insertion of the codon UGU into a nucleic acid sequence encoding the protein. Preferably the insertion of the codon for cysteine occurs at a pre-determined position within the nucleic acid sequence encoding the protein, for example at a location within that region of the nucleic acid sequence encoding the C-terminus (or carboxyl end) of the protein. Thereafter the modified protein can be expressed, for example in a cell expression system.

The term “protein” as used herein means, in general terms, a plurality of amino acid residues joined together by peptide bonds. It is used interchangeably and means the same as peptide and polypeptide.

The term “protein” is also intended to include fragments, analogues and derivatives of a protein wherein the fragment, analogue or derivative retains essentially the same biological activity or function as a reference protein.

The protein may be a linear structure but is preferably a non-linear structure having a folded, for example tertiary or quaternary, conformation. The protein may have one or more prosthetic groups conjugated to it, for example the protein may be a glycoprotein, lipoprotein or chromoprotein. Preferably the protein is a complex protein.

Preferably the protein comprises between 10 and 1000 amino acids, for example between 10 and 600 amino acids, such as between 10 and 200 or between 10 and 100 amino acids. Thus the protein may comprise between 10 and 20, 50, 100, 150, 200 or 500 amino acids.

In a preferred aspect of the invention the protein has a molecular weight greater than 10 kDa. The protein may have a molecular weight of at least 20 kDa or at least 60 kDa, for example between 10 and 100 kDa.

The protein may belong to the group of fibrous proteins or globular proteins. Preferably, the protein is a globular protein.

Preferably the protein is a biologically active protein. For example, the protein may be selected from the group consisting of glycoproteins, serum albumins and other blood proteins, hormones, enzymes, receptors, antibodies, interleukins and interferons.

Examples of proteins may include growth factors, differentiation factors, cytokines e.g. interleukins, (eg. M-1, IL-2, IL-3, IL-4. IL-5, IL-6, IL-7. IL-8, IL-9, IL-10, IL-11. IL-12, IL-13, IL-14, IL-15, IL-16, IL-17, IL-18, IL-19, IL-20 or IL-21, either [alpha] or [beta]), interferons (eg. IFN-[alpha], IFN-[beta] and IFN-[gamma]), tumour necrosis factor (TNF), IFN-[gamma] inducing factor (IGIF), bone morphogenetic protein (BMP); chemokines, trophic factors; cytokine receptors; free-radical scavenging enzymes.

In a preferred aspect of the invention the protein is a hormone. Preferably the hormone is erythropoietin (Epo).

The protein modified by the method of the invention beneficially retains inherent protein function/activity.

In a further preferred aspect of the invention the protein is an enzyme. Preferably the enzyme is Glucosylceramidase (D-glucocerebrosidase) (Cerezyme™) or Sulfolobus solfataricus beta-glycosidase (SSbG).

The present invention is further based on the site selective introduction of a tag, such as an alkyne, azide or thiol group, into the side chain of an amino acid at a predetermined site within the amino acid sequence of a protein (as discussed hereinbefore) followed by sequential, and orthogonal, glycosylation reactions that are selective for each respective tag. In this way, differential multi-site chemical protein glycosylation is achieved.

Thus in a second aspect of the invention there is provided a method for glycosylating a protein wherein the method comprises the steps of
i) modifying a protein according to the method of the first aspect of the invention; and
ii) reacting the modified protein in (i) with
   (a) a carbohydrate moiety modified to include an azide group; and/or
   (b) a carbohydrate moiety modified to include an alkyne group in the presence of a Cu(I) catalyst.

As used herein “glycosylation” refers to the general process of addition of a glycosyl unit to another moiety via a covalent linkage.

Typically, where the protein is modified in step (i) to include an alkyne group, the reaction in step (ii) is with the carbohydrate moiety in (a). Moreover, where the protein is modified in step (i) to include an azide group, the reaction in step (ii) is with the carbohydrate moiety in (b).

Preferably the modification to the protein (step i) additionally comprises the step of modifying the protein as defined herein to include a thiol group, for example through the insertion of a cysteine residue.

In a preferred aspect of the invention there is provided a method of glycosylating a protein, the method comprising the steps of
i) (a) modifying a protein to include an alkyne and/or an azide group; and
   (b) before or after the modification to the protein in (a), optionally modifying a protein to include a thiol group ; and
ii) sequential reaction of the protein modified in (i) with a carbohydrate moiety (c) in the presence of a Cu (I) catalyst before or after reaction with a thiol-selective carbohydrate reagent (d)
   (c) a carbohydrate moiety modified to include an azide group and/or a carbohydrate moiety modified to include an alkyne group; and
   (d) a thiol-selective carbohydrate reagent.

Steps (i) (a) and (b) are as described herein. Where the protein to be modified contains a cysteine residue, modification of the protein to include a thiol group may not be necessary. Alternatively, it may be desirable to include one or more thiol groups in addition to those already present in the protein.

The thiol-selective carbohydrate reagent may include any reagent which reacts with a thiol group in a protein to introduce a glycosyl residue linked to the protein via a disulfide bond. The thiol-selective carbohydrate reagent may include, but is not limited to, glycoalkanethiosulfonate reagent, for example glycomethanethiosulfonate reagent (glyco-MTS) (see WO00/01712 the content of which is incorporated in full herein), glycoselenylsulfide reagents (see WO2005/000862 the content of which is incorporated herein in their entirety) and the glycothiosulfonate reagents (see WO2005/000862 the content of which is incorporated herein in their entirety). Glycomethanethiosulfonate reagents are of the formula CH₃—SO₂—S-carbohydrate moiety.

The glycothiosulfonate and glycoselenylsulfide (SeS) reagents are generally of the formula I in WO2005/000862 (incorporated by reference herein). Specifically glycoselenylsulfide (SeS) reagents are of the formula R—S—X-carbohydrate moiety wherein X is Se and R is an optionally substituted C1-10 alkyl, phenyl, pyridyl or napthyl group. Glycothiosulfonate reagents are of the formula R—S—X-carbohydrate moiety wherein X is SO₂ and R is an optionally substituted phenyl, pyridyl or napthyl group. Such reagents provide for site-selective attachment of the carbohydrate to the protein via a disulphide bond.

Preferably the carbohydrates to be modified include monosaccharides, disaccharides, trisaccharides, tetrasaccharides oligosaccharides and other polysaccharides, and include any carbohydrate moiety which is present in naturally occurring glycoproteins or in biological systems. Included are glycosyl or glycoside derivatives, for example glucosyl, glucoside, galactosyl or galactoside derivatives. Glycosyl and glycoside groups include both α and β groups. Suitable carbohydrate moieties include glucose, galactose, fucose, GlcNAc, GalNAc, sialic acid, and mannose, and polysaccharides comprising at least one glucose, galactose, fucose, GlcNAc, GalNAc, sialic acid, and/or mannose residue.

Carbohydrate moieties may include Glc(Ac)₄β-, Glc(Bn)₄β-, Gal(Ac)₄β-, Gal(Bn)₄β-, Glc(Ac)₄α(1,4)Glc(Ac)₃α(1,4)Glc(Ac)₄β-, β-Glc, β-Gal, α-Man, α-Man(Ac)₄, Man(1,6)Manα-, Man(1-6)Man(1-3)Manα-, (Ac)₄Man(1-6)(Ac)₄Man(1-3)(AC)₂Manα-, -Et-β-Gal, -Et-β-Glc, Et-α-Glc, -Et-α-Man, -Et-Lac, -β-Glc(Ac)₂, -β-Glc(Ac)₃, -Et-α-Glc(Ac)₂, -Et-α-Glc(Ac)₃, -Et-α-Glc(Ac)₄, -Et-β-Glc(Ac)₂, -Et-β-Glc(Ac)₃, -Et-β-Glc(Ac)₄, -Et-α-Man(Ac)₃, -Et-α-Man(Ac)₄, -Et-β-Gal(Ac)₃, -Et-β-Gal(Ac)₄, -Et-Lac(Ac)₅, -Et-Lac(Ac)₆, -Et-Lac(Ac)₇, and their deprotected equivalents.

Any saccharide units making up the carbohydrate moiety which are derived from naturally occurring sugars will each be in the naturally occurring enantiomeric form, which may be either the D-form (e.g. D-glucose or D-galactose), or the L-form (e.g. L-rhamnose or L-fucose). Any anomeric linkages may be α- or β-linkages.

In one embodiment of the invention, carbohydrates that have been modified to include an azide group are glycosyl azides.

In one embodiment of the invention, carbohydrates that have been modified to include an alkyne group are alkynyl glycosides.

Preferably the azido and/or alkyne-modified carbohydrate moieties (e.g glycosyl azide and/or alkynyl glycoside) do not include a protecting group i.e. are unprotected. The unprotected azido and/or alkyne-modified carbohydrate moieties may be prepared by the addition of the azide or alkyne group to a protected sugar. Suitable protecting groups for any -OH groups in the carbohydrate moiety include acetate (Ac), benzyl (Bn), silyl (for example tert-butyl dimethylsilyl (TBDMSi) and tert-butyldiphenylsilyl (TMDPSi)), acetals, ketals, and methoxymethyl (MOM). The protecting group is then removed before or after attachment of the carbohydrate moiety to the protein. In this way, the reaction defined in step (ii) is carried out with an unprotected glycoside.

In a preferred aspect of the invention the Cu(I) catalyst is CuBr or CuI. Preferably the catalyst is CuBr. The Cu(I) catalyst may be provided by the use of a Cup salt (e.g. Cu(II)SO₄) in the reaction which is reduced to Cu(I) by the addition of a reducing agent (e.g. ascorbate, hydroxylamine, sodium sulfite or elemental copper) in situ in the reaction mixture. Preferably the Cu(I) catalyst is provided by the direct addition of Cu(I)Br to the reaction. Preferably the Cu(I)Br is provided at high purity, for example at least 99% purity such as 99.999%. Preferably still the Cu(I)catalyst (e.g.Cu(I)Br) is provided in a solvent in the presence of a stabilising ligand e.g.a nitrogen base. The ligand stabilizes Cu(I) in the reaction mixture; in its absence oxidation to Cu(II) occurs rapidly. Preferably the ligand is tristriazolyl amine ligand (Wormald and Dwek, Structure, 7, R155-R160 (1999)). The solvent for the catalyst may have a pH of 7.2-8.2. The solvent may be a water miscible organic solvent (e.g tert-BuOH) or an aqueous buffer such as phosphate buffer. Preferably the solvent is acetonitrile.

The reaction in step (ii) is a [3+2] cycloaddition reaction between an alkyne group (on the protein and/or the glycoside) and an azide group (on the protein and/or glycoside) to generate substituted 1,2,3-triazoles (Huigsen, Proc. Chem. Soc. 357-369 (1961)) which provide a link between the protein and the sugar(s).

A further aspect of the invention provides a protein modified by the method of the first or second aspect of the invention.

A further aspect of the invention provides a protein of formula (I), (II) or (III)

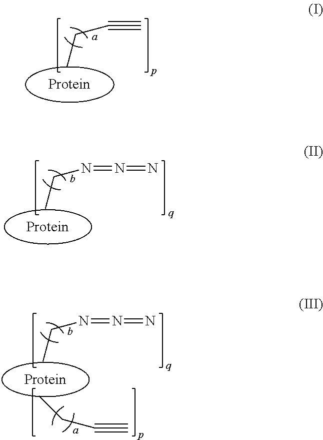

wherein a and b are integers between 0 and 5 (e.g. 0, 1, 2, 3, 4 or 5); p and q are integers between 1 and 5 (e.g. 1, 2, 3, 4 or 5); and wherein the protein is as defined herein.

A yet further aspect of the invention provides a glycosylated protein modified by the method of the second aspect of the invention.

The invention further provides a glycosylated protein of formula (IV)

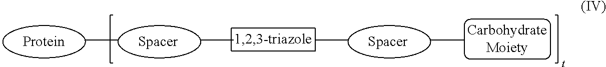

wherein t is an integer between 1 and 5 (e.g. 1, 2, 3, 4 or 5); and the spacer, which may be absent, is an aliphatic moiety having from 1 to 8 C atoms.

In a preferred aspect of the invention the spacer is a substituted or unsubstituted C1-6 alkyl group. Preferably the spacer is absent, methyl or ethyl.

In a further preferred aspect of the invention the spacer is a heteroalkyl wherein the heteroatom is O, N or S and the alkyl is methyl or ethyl. Preferably the heteroalkyl group is of formula CH₂(X)_{y} wherein X is O, N or S and Y is 0 or 1. Typically the heteroatom is directly linked to the carbohydrate moiety.

A substituent is halogen or a moiety having from 1 to 30 plural valent atoms selected from C, N, O, S and Si as well as monovalent atoms selected from H and halo. In one class of compounds, the substituent, if present, is for example selected from halogen and moieties having 1, 2, 3, 4 or 5 plural valent atoms as well as monovalent atoms selected from hydrogen and halogen. The plural valent atoms may be, for example, selected from C, N, O, S and B, e.g. C, N, S and O.

The term “substituted” as used herein in reference to a moiety or group means that one or more hydrogen atoms in the respective moiety, especially 1, 2 or 3 of the hydrogen atoms are replaced independently of each other by the corresponding number of the described substituents.

It will, of course, be understood that substituents are only at positions where they are chemically possible, the person skilled in the art being able to decide (either experimentally or theoretically) without inappropriate effort whether a particular substitution is possible. For example, amino or hydroxy groups with free hydrogen may be unstable if bound to carbon atoms with unsaturated (e.g. olefinic) bonds. Additionally, it will of course be understood that the substituents described herein may themselves be substituted by any substituent, subject to the aforementioned restriction to appropriate substitutions as recognised by the skilled man.

Substituted alkyl may therefore be, for example, alkyl as last defined, may be substituted with one or more of substituents, the substituents being the same or different and selected from hydroxy, etherified hydroxyl, halogen (e.g. fluorine), hydroxyalkyl (e.g. 2-hydroxyethyl), haloalkyl (e.g. trifluoromethyl or 2,2,2-trifluoroethyl), amino, substituted amino (e.g. N-alkyllamino, N,N-dialkylamino or N-alkanoylamino), alkoxycarbonyl, phenylalkoxycarbonyl, amidino, guanidino, hydroxyguanidino, formamidino, isothioureido, ureido, mercapto, acyl, acyloxy such as esterified carboxy for example, carboxy, sulfo, sulfamoyl, carbamoyl, cyano, azo, nitro and the like.

In a preferred aspect of the invention, the glycosylated protein is of formula (V)

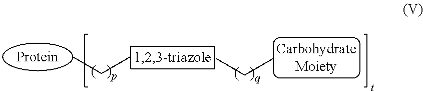

wherein p and q are integers between 0 and 5 (e.g. 0, 1, 2, 3, 4 or 5); t is an integer between 1 and 5 (e.g. 1, 2, 3, 4 or 5); and wherein the protein and carbohydrate moiety are as defined herein.

The protein or the carbohydrate moiety may be linked to the 1,2, 3,-triazole at position 1 or 2 as shown in formula (VI) and (VII) below. Thus the glycosylated protein of the invention may be of formula (VI) or (VII)

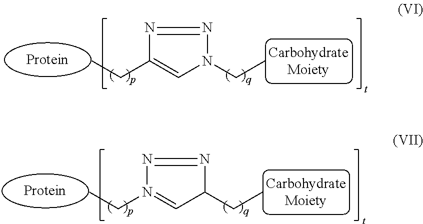

wherein the protein, carbohydrate moiety p, q and t are as defined herein.

Preferably p is 2.

Preferably q is O.

The invention further provides a glycosylated protein of formula (VIII)

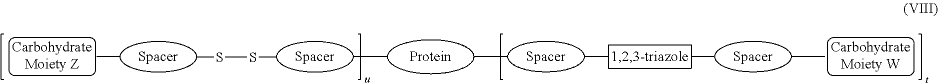

wherein u is an integer between 1 and 5 (e.g. 1, 2, 3, 4 or 5); the spacer and t are as defined herein and wherein W and Z are carbohydrate moieties that may be the same or different.

Preferably the glycosylated protein is of formula (IX)

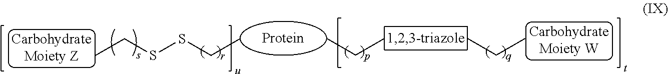

wherein the spacer, p, q, t and u are as defined herein; and wherein r and s are integers between 0 and 5 (e.g. 0, 1, 2, 3, 4 or 5).

Preferably still the glycosylated protein is of formula (X) or (XI)

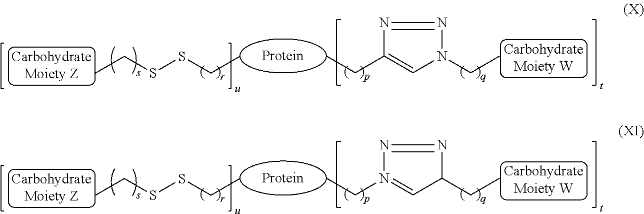

wherein the protein, spacer, carbohydrate moieties, p, q, r, s, t and u are as defined herein.

The glycosylated proteins of the present invention typically retain their inherent function and certain proteins may demonstrate an improvement in function, for example increased enzyme activity (relative to the un-glycosylated enzyme) following glycosylation as described herein. The glycosylated proteins of the invention may also show additional protein-protein binding capabilities with other different proteins, for example lectin binding capability. Thus the method of the present invention is useful in manipulating protein function for example to include additional, non-inherent, protein functionality such as protein-protein binding capabilities with other different proteins e.g. lectins.

The glycosylated proteins of the invention may be useful in medicine, for example in the treatment or prevention of a disease or clinical condition. Thus the invention provides a pharmaceutical composition comprising a glycosylated protein according to the invention in combination with a pharmaceutically acceptable carrier or diluent. The proteins of the invention may be useful in, for example, the treatment of anaemia or Gaucher's disease.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

The invention will now be described with reference to the following non-limiting Examples.

### EXAMPLES

Multi Site-Directed Mutagenesis:

A number of mutants of the β-galactosidase SsβG were created using the QuikChange Multi Site-Directed Mutagenesis Kit commercially available from Stratagene [catalog no. 200514]. Plasmid pET28d carrying SsβG C344S was used as a template¹. The corresponding mutagenic primers were designed for replacement of Met residues by Ile and were custom synthesized by Sigma-Genosys and were as follows:

TABLE S1

| | Sequence of primer (all mutagenic |
|---|---|
| Mutation | primers are 5′ phosphorylated) |
| M21I | TGACCCTGGTGTTCCTATTTCTGATTGAAATCCGG |
| M43I | CTTACTAATCCCGCTGCTATGTTTTCTGGATCATGAACC |
| M73I | CATTTAGTCTAGCTATTTTTAATCCTATTTTTTGTGCAT |
| | TATCGTGAAATGTC |
| M1481 | TAGAGGTAATGGCCAATGATAGATGTTTAGTATAAAGTA |
| | AAGTCCTC |
| M2041 | CCAACAACGTTAGGTTCATTTATTGTTGAGTACTCATCC |
| | AC |
| M236I | GAGCTTGAATGATGTTATATATCGCCCTACGGGAAAG |
| M275I | CCATCTCTACCGCTTCTATATCTTTATCCGTTAACGG |
| M280I | CATCTATTATCATTTTCAGCGATCTCTACCGCTTCTATA |
| | TC |
| M3831 | CAATACCATTTTCAGTAACGTAGATATAGAGATGATATC |
| | TATTCCAG |
| M439I | CCTTTAACAGACCAAACCTTATAGAGAATCCTGAAGCCC |

In this way mutants with a desired number (between 1 and 10) of Met residues could be introduced. Further mutations were introduced by single site-directed mutagenesis using sets of complementary forward and reverse mutagenic primers:

TABLE S2

| | Sequence of primer (all mutagenic |
|---|---|
| Mutation | primers are 5′ phosphorylated) |
| I439C | forward: |
| | GAATGGGCTTCAGGATTCTCTTGCAGGTTTGGTCTGTTA |
| | AAGGTC |
| | reverse: |
| | GACCTTTAACAGACCAAACCTGCAAGAGAATCCTGAAGC |
| | CCATTC |

The corresponding mutant proteins could be expressed using the protocol outlined below.

Protein Expression with Met Analogue Incorporation:

Incorporation of homopropargyl glycine (Hpg) or azido homoalanine (Aha) into proteins by protein expression using medium shift protocol². An overnight culture of Escherichia coli B834 (DE3), pET28d SsβG C344S was grown in molecular dimensions medium (˜16 h) supplemented with kanamycin (50 μg/mL) and L-methionine (40 μg/mL). The overnight culture was used to inoculate pre-warmed (37° C.) culture medium (1.0 L, same composition as above) and the cells were grown for 3 h (0D₆₀₀ ˜1.2). The medium shift was performed by centrifugation (6,000 rpm, 10 min, 4° C.), resuspension in methionine-free medium (0.5 l) and transfer into pre-warmed (37° C.) culture medium (1.0 L) containing the unnatural amino acid (DL-Hpg at 80 μg/mL, L-Aha at 40 μg/mL). The culture was shaken for 15 min at 29° C. and then induced by addition of IPTG to 1.0 mM. Protein expression was continued at 29° C. for 12 h.

The culture was centrifuged (9,000 rpm, 15 min, 4° C.) and the cell pellets frozen at −80° C. The protein was purified by nickel affmity chromatography: The cell pellets were transferred into binding buffer (50 ml) and cells were broken up by sonication (3*30 s, 60% amplitude) and the suspension was centrifuged (20,000 rpm, 20 min, 4° C.). The supernatant was filtered (0.8 μm) and the protein was purified on a nickel affinity column eluting with an increasing concentration of imidazole. Elution was monitored by LTV absorbance at 280 nm and fractions combined accordingly. The combined fractions were dialyzed (MWCO 12-14 kDa) at 22° C. overnight against sodium phosphate buffer (50 mM, pH 6.5, 4.01). The protein solution was filtered (0.2 μm) and stored at 4° C.

Synthesis of Reagents.

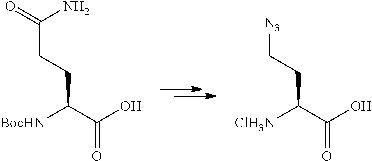

L-Homoazido alanine was synthesized via a Hofmann-rearrangement, diazotransfer, and deprotection strategy as described in literature³.

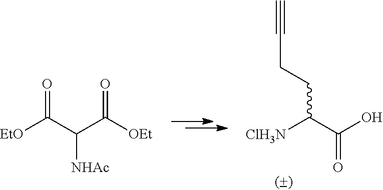

DL-Homopropargyl glycine was prepared from diethyl acetamidomalonate by homopropargyl alkylation, hydrolysis, and decarboxylation as described previously².

### 1-azido-2-acetimido-2-deoxy β-D-glucopyranoside 1

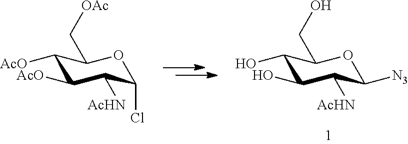

N-Ac-glucosyl azide were synthesised from the corresponding acetyl protected glycosyl chloride followed by Zemplén deacetylation⁴.

### Chitobiosyl Azide 2

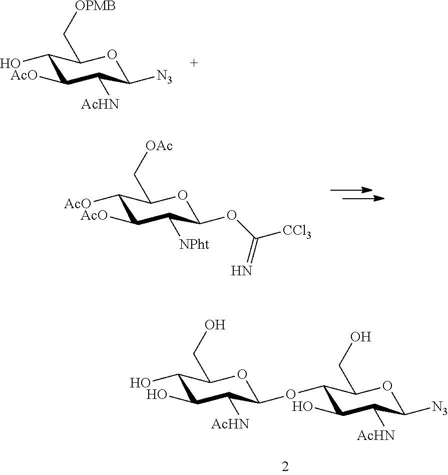

Chitobiosyl azide was prepared as described by Macmillan et al⁵.

### (2-methanethiosulfonate-ethyl) α-D-glucopyranoside 7

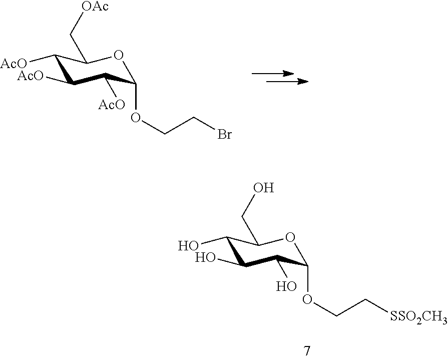

α-Glucopyranosyl MTS-reagent was prepared from known bromide via protecting group removal and methanethiosulfonate substitution as described in ref⁶.

### (2-azido-ethyl) α-D-mannopyranoside 3

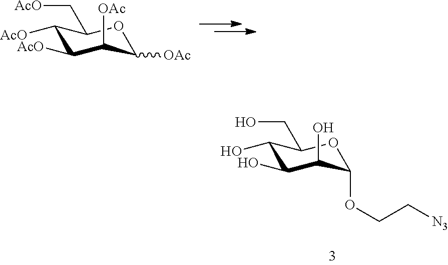

Azidoethyl α-mannopyranoside 3 was synthesized according to literature procedures from mannose pentaacetate by glycosylation with bromoethanol followed by azide substitution^{6,7}.

### Tris-triazole Ligand 11

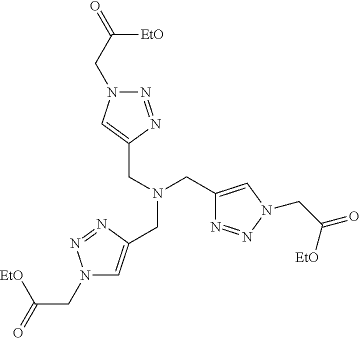

Tris-triazole ligand 11 was prepared from azido ethyl acetate and tripropargyl amine as described⁸.

### Ethynyl C-galactoside 5

Ethynyl β-C-galactoside was prepared in the same manner as the known C-glucoside according to the method of Xu, Jinwang; Egger, Anita; Bernet, Bruno; Vasella, Andrea; Hely. Chim. Acta; 79 (7), 1996, 2004-2022.

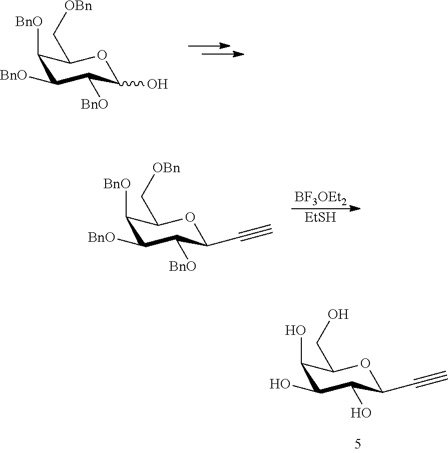

Small Molecule Model Glyco-CCHA Reactions

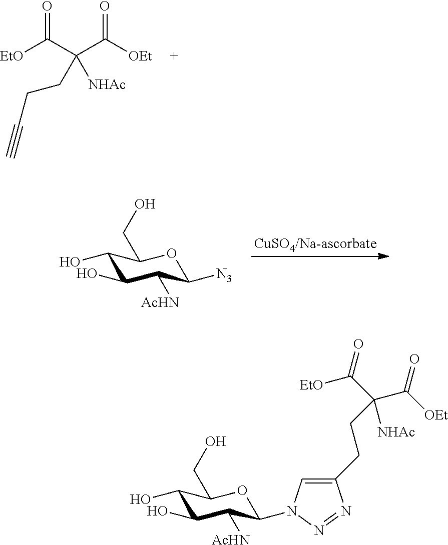

Diethyl homopropargyl acetamidomalonate (55 mg, 0.20 mmol), HO₃GlcNAc-N₃ 1 (101 mg, 0.41 mmol), sodium ascorbate (202 mg, 10 mmol) and tris-triazoleyl amine ligand 11 (6 mg, 0.012 mmol) were dissolved in a mixture of MOPS buffer (pH 7.5, 0.2 M; 4.0 mL) and tert-butyl alcohol (2.0 mL). Copper(II)sulfate solution (0.1 M, 100 μL, 0.01 mmol) was added to the stirred solution and the reaction mixture was stirred for 28 h at room temperature. The solvent was then evaporated under reduced pressure and the remaining residue was purified by flash column chromatography on (silica, AcOEt to 15% MeOH in AcOEt). The product appeared as a colorless film (83 mg, 79%).

### Methyl (S)-2-[N-acetyl-amino]-4-{1-(2-deoxy-N-acetylamino-β-D-glucopyranosyl)[1,2,3,]triazol-4-yl}butanoate

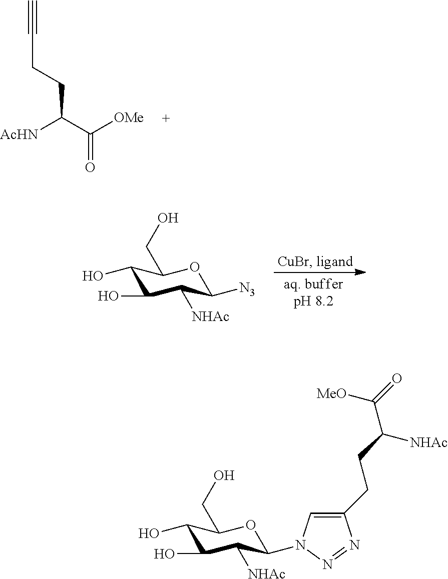

Cuprous bromide (10 mg, 0.070 mmol) was dissolved in acetonitrile (1 mL) and ligand (0.58 mL of a 0.12 M solution in acetonitrile) added. This solution (38 μL, 5% catalyst loading) was added to a solution of alkyne amino acid (15 mg, 0.08 mmol) and sugar 2 (31 mg, 0.13 mmol) in sodium phosphate buffer (0.5 mL, 0.15M, pH 8.2). The reaction mixture was stirred under argon at room temperature for 1 hr, after which TLC-analysis indicated disappearance of alkyne starting material. The mixture was diluted with ethyl acetate and washed with water (10 mL) and the aqueous layer washed with AcOEt. The aqueous layer was evaporated to dryness under reduced pressure. The residue was purified by column chromatography (silica, 1:1 ethyl AcOEt/iPrOH to 4:4:2 H₂O/iPrOH/AcOEt) to afford the desired 1,2,3-triazole (26 mg, 74%) as a colourless glassy solid.

### Methyl (S)-2-[N-acetyl-amino]-4-{4-(β-D-galactopyranosyl)[1,2,3]triazol-1-yl}butanoate

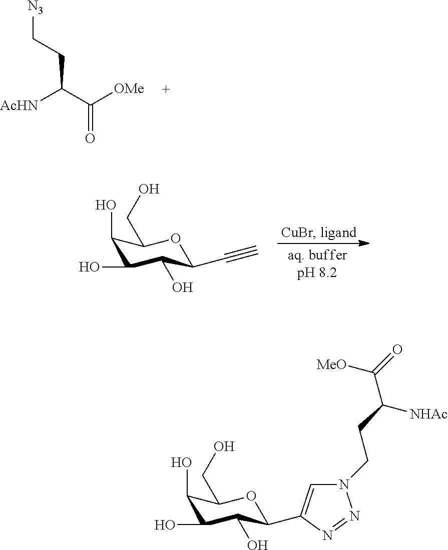

Cuprous bromide (10 mg, 0.070 mmol) was dissolved in acetonitrile (1 mL) and tristriazolyl amine ligand (0.58 mL, 0.12 M in acetonitrile) was added. This solution (45 μL, 5% catalyst loading) was added to a solution of amino acid (20 mg, 0.10 mmol) and sugar 5 (28 mg, 0.13 mmol) in sodium phosphate buffer (0.5 mL, 0.15 M, pH 8.2). The reaction mixture was stirred under argon at room temperature for 3 hr. The reaction mixture was evaporated to dryness under reduced pressure and the residue purified by column chromatography (silica, 9:1 AcOEt/MeOH to 4:4:2 H₂O/iPrOH/AcOEt) to afford the desired 1,2,3-triazole (37 mg, 97%) as a white solid.

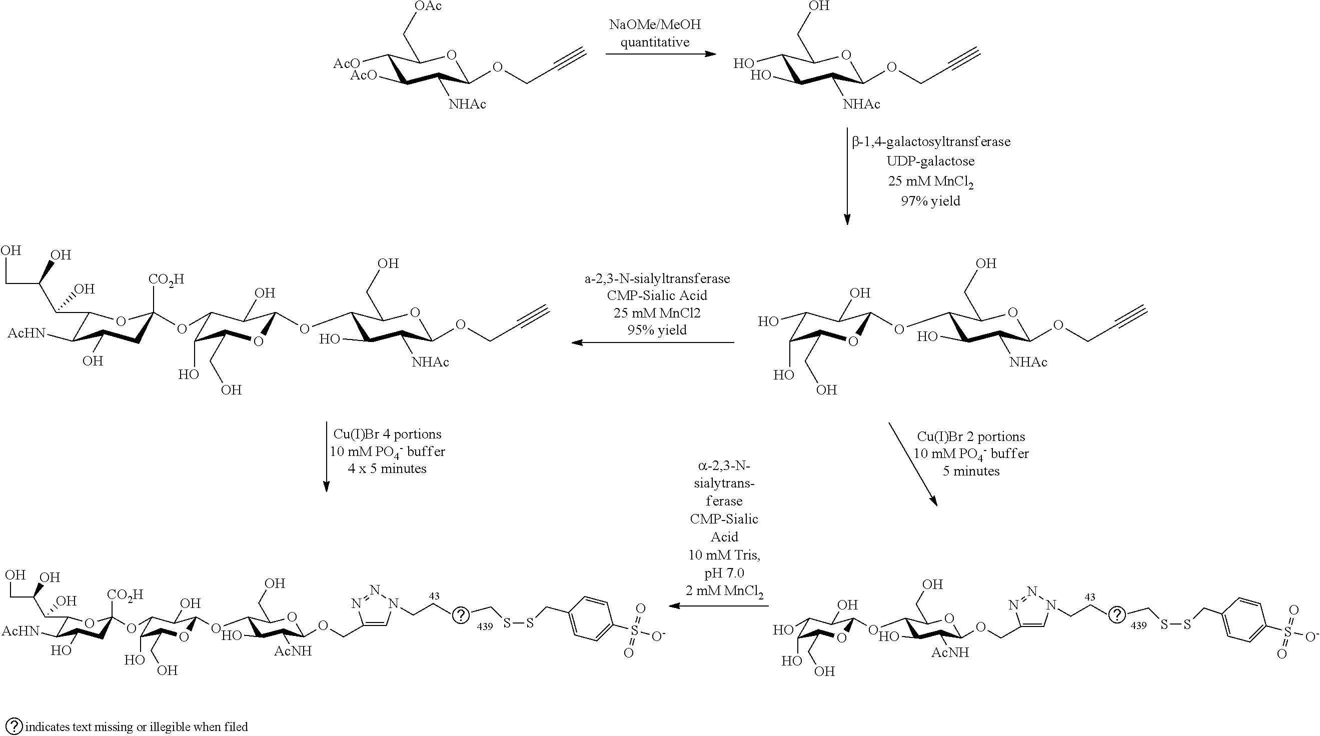

figure xx: synthesis of O-propargyl SiaLacNAc from O-propargyl-N-acetylglucosamine A very simple high-yielding enzymatic synthesis of SiaLacNAc was employed (reference Baisch, et. al.). At no stage purification other than flash column chromatography was required to obtain any of the products.

### 2-acetamido-2-deoxy-1-propargyl-β-D-glucopyranoside

2-Acetamido-2-deoxy-1-propargyl-b-D-glucopyranoside has been described previously. For our purposes it was prepared as shown below according to the method of Vauzeilles, Boris; Dausse, Bruno; Palmier, Sara; Beau, Jean-Marie; Tetrahedron Lett., 42(43) 2001, 7567-7570

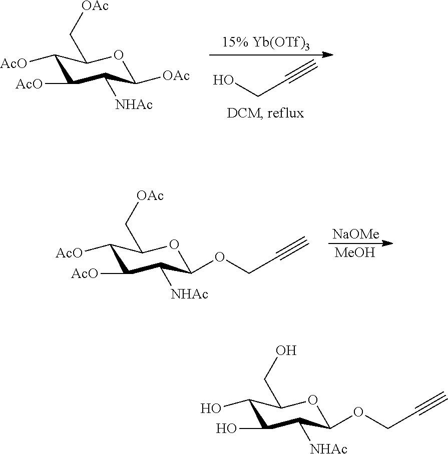

### 2-Acetamido-2-deoxy-4-O-β-d-galactopyranosyl-1-propargyl-D-glucopyranoside

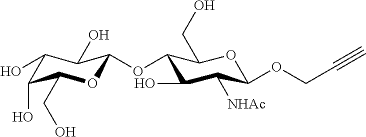

2-Acetamido-2-deoxy-1-propargyl-β-D-glucopyranoside (15.0 mg, 0.058 mmol) and uridine-5′-diphosphogalactose disodium salt (59 mg, 0.092 mmol) were dissolved in 1.0 mL of sodium cacodylate buffer (0.1 M, 25 mM MnCl₂, 1 mg/mL bovine serum albumin, pH 7.47). β-1,4-galactosyltransferase (ec 2.4.1.22, 0.8 U) and alkaline phosphatase (ec 3.1.3.1, 39 U) were added and the mixture was shaken gently at 37° C. for 21 hours when tic (1:2:2 water:isopropyl alcohol:ethyl acetate) indicated the complete disappearance of the acceptor sugar (R_{f} 0.8). The reaction mixture was lyophilised onto silica and purified by flash column chromatography (2:5:6 water:isopropyl alcohol:ethyl acetate) to yield 23.7 mg (97% yield) of a white amorphous solid.

### Propargyl-(5-acetimido-3,5-dideoxy-d-glycero-α-D-galacto-2-nonulopyranosylonic acid-(2→3)-β-D-galactopyranosyl-(1→4)-2-acetimido-2-deoxy-β-D-glucopyranoside

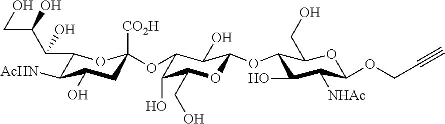

2-Acetamido-2-deoxy-4-O-β-d-galactopyranosyl-1-propargyl-D-glucopyranoside (12 mg, 0.028 mmol) was dissolved in 1.4 mL of water. Sodium cacodylate was added (60 mg, 0.28 mol, final concentration: 0.2 M), as were manganese chloride tetrahydrate (8 mg, 0.041 mmol, final concentration 29 mM) and bovine serum albumin (2 mg). The pH was adjusted to 7.1 prior to the addition of cytidine-5′-monophospho-N-acetylneuraminic acid sodium salt (19.8 mg, 1 equivalent) and α-2,3-(N)-sialyltransferase, recombinant ex. Spodoptera frugiperda, ec 2.4.99.6, 30 mU) and alkaline phosphatase (ec 3.1.3.1, 30 U) were added and the mixture was gently shaken at 37° C. for 70 hours, after which the reaction mixture was lyophilised onto silica and purified by flash column chromatography (5:11:15 water:isopropyl alcohol:ethyl acetate) to yield 20.9 mg of an amorphous solid (95% yield).

ELISA Assay for Measuring Role of Sulfotyrosine in P-Selectin Binding

Experiments were carried out to show that proteins glycosylated by the invention have altered biological binding properties.

The ELISA assay was modified from the assay published previously.

The modified SsβG proteins were coated at 200 ng/well (NUNC Maxisorp, 2 μg/mL, 50 mM carbonate buffer, pH 9.6).

Dithiothreitol (5 μL/well, 50 mg/mL in water) was added to reduce off the sulfated tyrosine mimic to the appropriate lanes. The plate was incubated at 4° C. for 15 hours.

The wells were blocked with bovine serum albumin (25 mg/mL in assay buffer: 2 mM CaCl2, 10 mM Tris, 150 mM NaCl, pH 7.2, 200 μL per well) for 2 hours at 37° C.

The plate was washed with washing buffer (assay buffer containing 0.05% v/v Tween 20, 3×400 μL per well), prior to addition of P-selectin (ex Calbiochem, cat. no. 561306, recombinant in CHO-cells, truncated sequence, transmembrane and cytoplasmic domain missing, serial double dilution from 400 ng/well to 1.6 ng/well for each of the differently modified SsβG mutants in 100 μL of washing buffer). The plate was incubated at 37° C. for 3 hour.

After washing with washing buffer twice, the wells were incubated with anti-P-selectin antibody (IgG1 subtype, ex Chemicon, clone AK-6, 100 ng/well in 100 μL assay buffer) for 1 hour at 21° C. (plus 3 control wells) and washed with washing buffer (3×300 μL/well).

Each of the wells was incubated with anti-mouse IgG-specific-HRP-conjugate (ex Sigma, A 0168) for 1 hour at 21° C. The wells were washed with washing buffer (3×300 μL). The binding was visualised by the addition of TMB-substrate solution (ex Sigma-Aldrich, T0440, 100 μL per well) and incubating in the dark at 22° C. until the absorbances read at 370 nm came in the linear range (approx. 15 minutes).

### (S)-2-amino-4-{4-(β-D-galactopyranosyl)[1,2,3]triazol-1-yl}butanoate

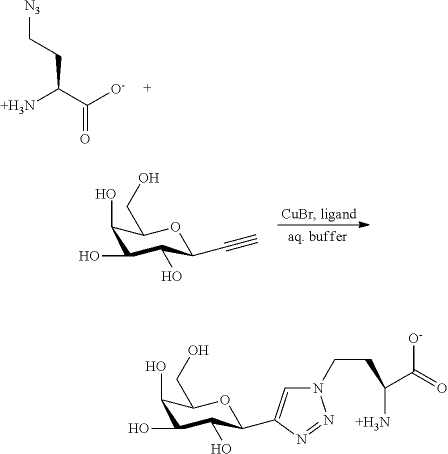

Using the same method as above an optimization study was conducted using 1.5 eq of Ethynyl C-galactoside 5 relative to Aha.

| pH | Conversion, %^{a} |
|---|---|
| 5.2 | 0 |
| 6.2 | 16 |
| 7.2 | 61 |
| 8.2 | 82 |
| 9.2 | 45 |
| no ligand, 8.2 | 7 |

^{a}As judged by ¹H NMR (D₂O, 500 MHz); isolated yield confirmed for pH 8.2 value at 84%

Tamm-Horsfall Fragment Preparation:

Tamm-Horsfall (THp) peptide fragment (295-306; H₂N-Gln-Asp-Phe-Asn-Ile-Thr-Asp-Ile-Ser-Leu-Leu-Glu-C(O)NH₂)¹² analogues (H₂N-Gln-Asp-Phe-Aha/Hpg-Ile-Thr-Asp-Ile-Cys-Leu-Leu-Glu-C(O)NH₂) were synthesized by means of Fmoc-chemsitry on Rink amide MBHA-polystyrene resin [1% divinyl benzene, Novabiochem cat. no. 01-64-0037] using a microwave assisted Liberty CEM peptide synthesizer.

Representative Procedure for Glyco-Cycloaddition of Azidoprotein Aha-Containing Protein:

Ethynyl-β-C-galactoside (5 mg, 0.027 mmol) 5 was dissolved in sodium phosphate buffer (0.5 M, pH 8.2, 200 μL). Protein solution (0.2 mg in 300 μL) was added to the above solution and mixed thoroughly. A freshly prepared solution of copper(I) bromide (99.999%) in acetonitrile (33 μL of 10 mg/mL) was premixed with an acetonitrile solution of tris-triazolyl amine ligand 11 (12.5 μL of 120 mg/mL). The preformed Cu-complex solution (45 μL) was added to the mixture and the reaction was agitated on a rotator for lh at room temperature. The reaction mixture was then centrifuged to remove any precipitate of Cu(II) salts and the supernatant desalted on a PD 10 column eluting with demineralised water (3.5 mL). The eluent was concentrated on a vivaspin membrane concentrator (10 kDa molecular weight cut off) and washed with 50 mM EDTA solution and then with demineralized water (3×500 μL). Finally, the solution was concentrated to 100 μL and the product was characterized by LC-MS, SDS-PAGE gel electrophoresis, CD, tryptic digest and tryptic digest-LC MS/MS.

TABLE S3
Tryptic digest-HPLC/MS data for example starting material
SSβG-Cys344Ser-Met21Aha-Met43-Aha-Met73Aha-Met148Aha-
Met204Aha-Met236Aha-Met275Aha-Met280Aha-Met383Aha-Met439Aha

| | | |
|---|---|---|
| Cleavage | | |
| fragment | Retention | Charge states (m/z) |

| [residue #] | time [min] | +1 | +2 | +3 | +4 |
|---|---|---|---|---|---|
| T2 16-41 | 24.1 | | 1459.1 | 973.1 | |
| T3 42-70 | 24.7 | | 1584.7 | 1056.8 | |
| T5 79-82 | 4.1 | 442.3 | | | |
| T16 147-168 | 32.2 | | | 930.5 | |
| T22 200-225 | 25.5 | | 1406.7 | 938.1 | |
| T25 241-251 | 17.7 | | 641.8 | | |
| T29 280-292 | 17.1 | | 757.3 | | |
| T45 427-446 | 26.8 | | 1193.0 | 795.7 | |

TABLE S4
Tryptic digest-HPLC/MS data for regioselectively
trigalactosylated SSβG-Cys344Ser-Met21Aha-Met43-
T-Gal-Met73Aha-Met148Aha-Met204Aha-Met236Aha-Met275-
T-Gal-Met280-T-Gal-Met383Aha-Met439Aha

| Cleavage | | | | | |
|---|---|---|---|---|---|
| fragment | retention | Charge states (m/z) | | | |
| [residue #] | time [min] | +1 | +2 | +3 | +4 |
| T2 16-41 | 24.4 | | 1459.6 | 973.4 | |
| T3Gal 42-70 | 23.1 | | | 1120.1 | 840.3 |
| T5 79-82 | 4.3 | 443.2 | | | |
| T22 200-225 | 25.6 | | 1407.1 | 938.4 | |
| T25 241-251 | 18.1 | 1282.7 | 641.8 | | |
| T29Gal₂ 280-292 | 6.9 | | 945.4 | 630.6 | |
| T45 427-446 | 26.7 | | 1193.5 | 796.0 | |

NB residues numbered here based on actual amino acids and include His-tag. The numbering used throughout the rest of this paper is based on WT sequence of SSβG. Thus, for example, tryptic fragment T29 #280-292 corresponds to 274-286 (K)D[TGal]EAVE[TGal]AENDNR(W).

Glyco-Cycloaddition of Alkynylprotein Hpg-Containing Protein:

An analogous procedure was employed for the modification of Hpg containing proteins. In this case an azide bearing carbohydrate (HO₃GlcNAcN₃) 1 was used as the reaction partner instead of the alkynyl-β-C-glycoside.

THp Fragment Dual Differential Glycoconjugation:

To a solution of freshly synthesized peptide (Hpg- or Aha-incorporated, 0.5 mg) in aqueous phosphate buffer (50 mM, pH 8.2, 0.3 mL) was added a solution of glucoside MTS-reagent 7 in water (50 μL, 33 mM, 5 eq.). The reaction was put on an end-over-end rotator for 1 hr before an aliquot underwent LCT-MS analysis using a Phenomenex Gemini 5μ C18 110A column (flow: 1.0 mL/min, mobile phase gradient: 0.05% formic acid in H₂O to 0.05% formic acid in MeCN over 20 min).

A solution of copper catalyst complex was made by dissolving cuprous bromide (5 mg, 99.999% pure) and tris-triazole ligand 11 (18 mg) in MeCN (0.5 mL). Ethynyl sugar 5 or azido sugar 1 (6 mg) was dissolved in the reaction mixture of the disulfide bond forming glycoconjugation before copper(I) complex (15 μL) was added. Reaction between Aha-displaying peptide and Ethynyl sugar was complete found by LC-MS analysis to be complete after 1 hr at rt. To the reaction of Hpg-displaying peptide and azidosugar an extra amount of copper(I) complex solution (10 μL) was added after 1 hr. After an additional period of 1 hr. LC-MS analysis demonstrated full conversion of starting material to the desired conjugated product. Reaction sites are marked with a circle:

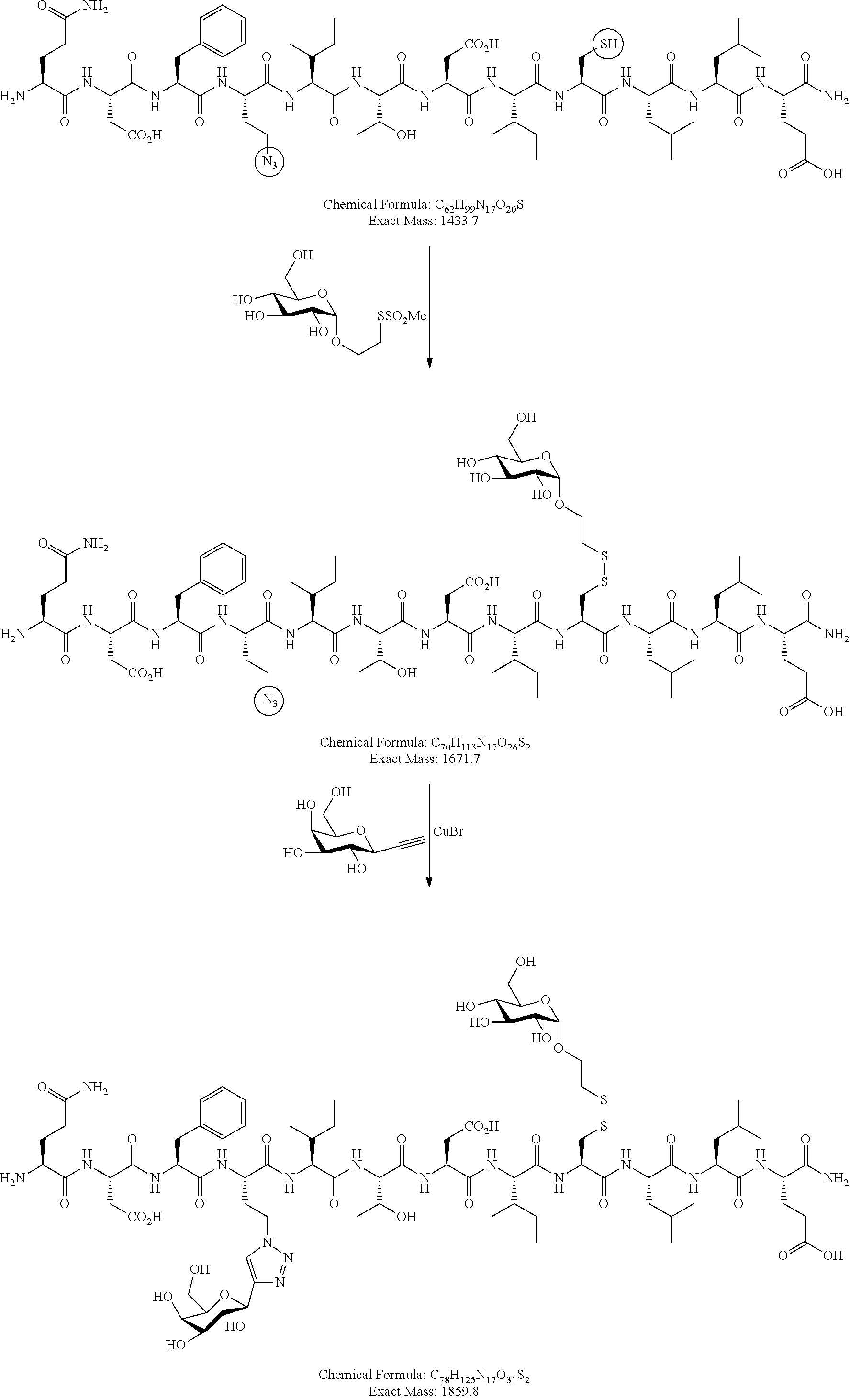

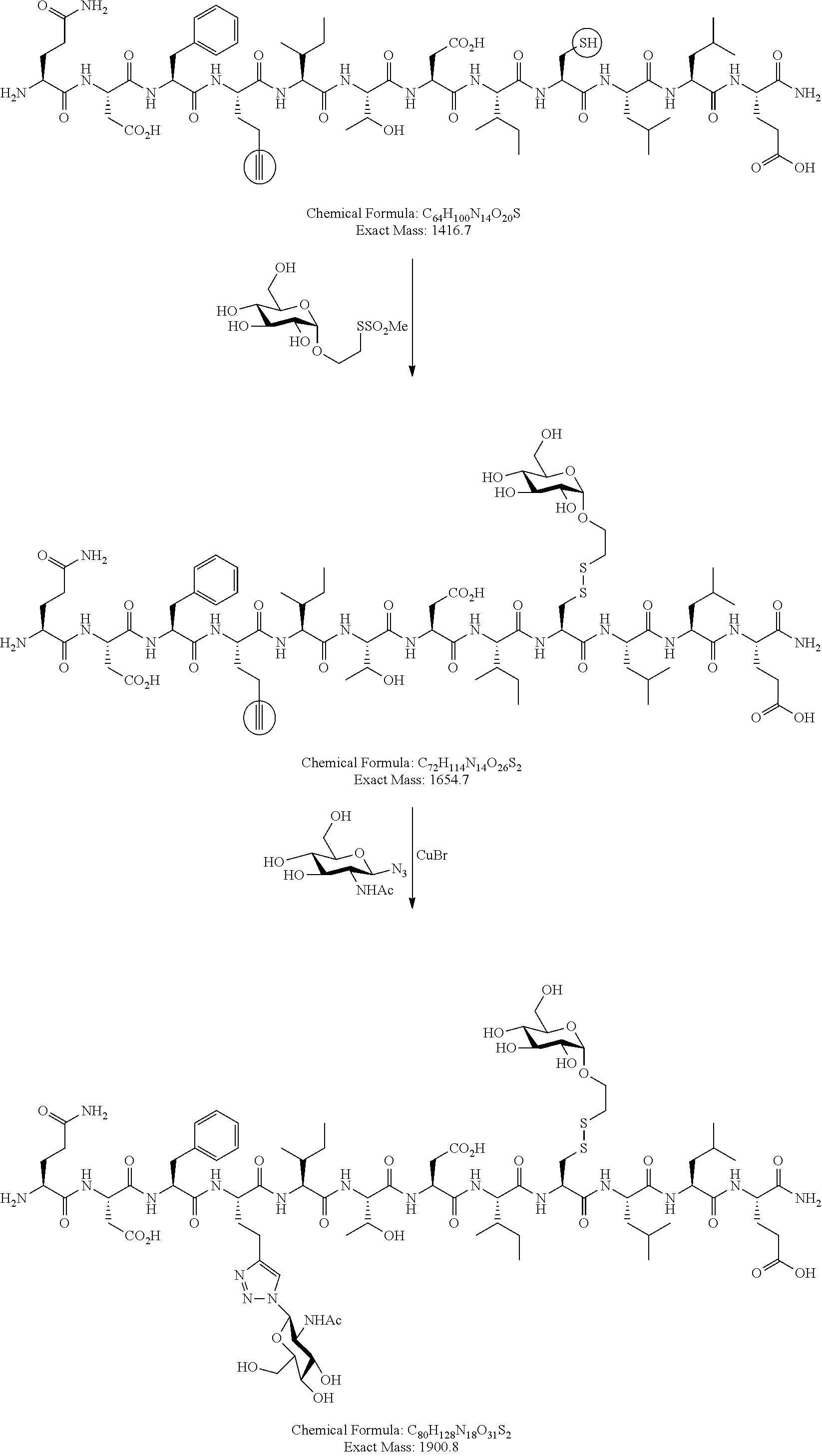

Comments on Optimization of Reaction Conditions for Glyco-Cycloaddition:

Tristriazole ligand 11 has been shown previously in the literature¹³ to be useful in stabilizing Cu(I) in the aqueous reaction mixture. In its absence, oxidation to Cu(II) occurs rapidly. Due to the low solubility of CuBr in other solvents, acetonitrile was chosen.

A slightly alkaline buffer system (pH 7.5-pH 8.5) was found to be most suited for the modification reaction. Many previous examples in the literature rely on in situ reduction of a Cu(II) salt by adding a reducing agent to the reaction mixture. All our attempts at employing in situ reduction of Cu(II) towards catalysis for protein modification proved unsatisfactory. The quality of spectra of the corresponding samples was low and deconvolution provided insufficient signal to noise ratio.

Enzyme Activity:

Kinetic analysis was carried out and showed that mutant proteins and glycoconjugates retain enzymatic activity (data not shown).

Lectin Binding Studies:

Experiments were carried out to show that the glycoconjugated sugars affect biological targeting

The lectin-binding properties¹⁵ of glycoconjugated SsβG mutants were characterized by retention analysis on immobilized lectin affinity columns [Galab cat no. PNA, Arachis hypogaea: 051061, Con A: 051041, Triticum vulgaris, K-WGA-1001]. Eluted fractions were visualized with Bradford reagent¹⁴ and absorption was determined at 595 nm.

TABLE S7

| | Non-glycosylated | Corresponding glycosylated |
|---|---|---|
| | SsβG | SsβG |
| PNA | 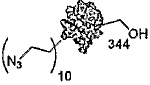 | 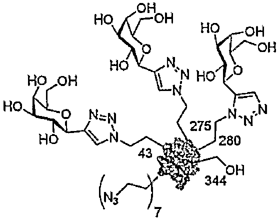 |
| Con A | 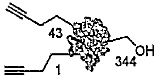 | 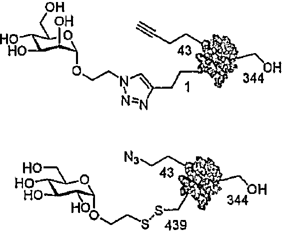 |
| WGA | 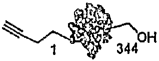 | 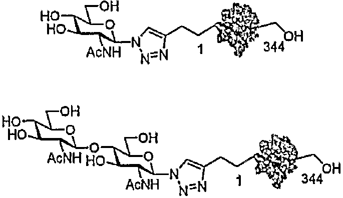 |

Man SsβG clearly demonstrated binding to legume lectin Concanavalin A (Con A) while Glc-conjugate (Glc SsβG) did not show significant binding above background. This was also found to be the case for β-Gal-triazole-conjugated SsβG binding to galactophilic lectin peanut agglutinin (PNA). Chitobiose (GlcNAc SsβG) conjugate, and to a small extend GlcNAc conjugates, however, were found to bind to wheat germ agglutinin (WGA) lectin, by retarding the neo-glycopeptides release of the spin affinity columns. The lack of binding of glucose-contrary to mannose conjugate, could possibly be explained by Con A's lower affinity for glucose¹⁶. Relative binding of monosaccharides by Con A has been found to be: MeaMan:Man:MeaGlu:Glu in the ratio 21:4:5:1. Mannose monosaccharides are hence bound 4 times tighter by Con A than glucose monosaccharide. The aromatic triazole may also contribute to increased binding of mannoside over disulfide linked glucoside¹⁷.

Lack of binding found in some and not others of the above mentioned constructs highlights the need for precise preparation of glycoproteins.

Solvent Accessibility:

Only a few studies of proteins reactivity in chemical reactions to date give an integrated assessment¹⁸ of amino acid residues accessibility¹⁹⁻²¹.

Protein crystal structure of SsβG was obtained from ref.²². The solvent accesibility for monomer A of dimeric dimer of SsβG was assessed by Naccess²³. Accessibility data for monomer B gave nearly identical values. The values given as relative total side-chain accessibility is of interest in this study. These are a measure of the accessibility of the side-chain of a given amino acid X relative to the accessibility of the same side-chain in the tripeptide Ala-X-Ala. Therefore, it is to be expected that the accessibility of N-terminal residue Met1 for the studied SsβG-mutants is even higher than for the calculated WT protein since the expressed mutants have Met1-Gly2 spaced from the rest of the sequence by a His₇-tag (not numbered).

Solvent accessibility was furthermore based on the natural amino acid sequence and not e.g. incorporated homoazidoalanine and homopropargyl glycine mutants.

The calculations were performed using different probe sizes (1.0 Å, 1.4 Å, and 2.8 Å). less amino acid side-chains become accessible by increasing the probe size.

Based on these data (see table below) it is to be anticipated that methionine residues at positions 1, 43, 275, 280 are relatively accessible. The same could be expected for their methionine analogue mutants.

TABLE S8

| Amino acid residue | 1.0 Å | 1.4 Å | 2.8 Å |
|---|---|---|---|
| Met1 | 54.2 | 53.2 | 59.9 |
| Met21 | 9.8 | 1.9 | 0.0 |
| Met43 | 48.0 | 46.6 | 48.3 |
| Met73 | 0.6 | 0.0 | 0.0 |
| Met148 | 3.6 | 0.0 | 0.0 |
| Met204 | 4.2 | 0.0 | 0.0 |
| Met236 | 17.3 | 11.2 | 2.1 |
| Met275 | 62.8 | 64.0 | 69.5 |
| Met280 | 37.5 | 35.1 | 26.6 |
| Cys344 | 6.6 | 2.0 | 0.0 |
| Met383 | 2.5 | 0.0 | 0.0 |
| Met439 | 12.3 | 7.5 | 3.4 |

The figure below shows, in colors, the relative accessibility of WT-SsβG.

On TIM Barrels:

The far most common tertiary fold observed in protein crystal structures is the TIM barrel. It is believed to be present in around 10% of all proteins²⁴.

On the Tamm-Horsfall (THp) Blycoprotein:

THp is the most abundant glycoprotein in mammals^{12,25} N- as well as O-Glycosylation pattern is known to play a key role in the biological function of Thp.²⁶ Of the eight possible N-glycosylation sites, seven are known to be glycosylated. Among these are Asn-298 residue.²⁷

### Glycosylation of Erythropoietin and Glucosylceramidase

For Erythropoietin the respective glycosylation sites are Asn24, Asn38 and Asn83 for the N-linked carbohydrates. The protein contains a single O-linked glycosylation site at Ser126. Using multi site-directed mutagenesis and incorporation of inethione analogs at the newly introduced Met sites (the natural sequence of Epo contains only a single methionine (M54) the protein can be modified.

Glucosylceramidase (D-glucocerebrosidase), a 60 kD glycoprotein which plays an important role in the development of Gaucher's disease, represents is also glycosylated by this methodology.

### REFERENCES

1. Hancock, S. M., Corbett, K., Fordham-Skelton, A. P., Gatehouse, J. A. & Davis, B. G. Developing promiscuous glycosidases for glycoside synthesis: Residues W433 and E432 in Sulfolobus solfataricus beta-glycosidase are important glucoside- and galactoside-specificity determinants. Chem Bio Chem 6, 866-875 (2005).
2. van Hest, J. C. M., Kiick, K. L. & Tirrell, D. A. Efficient incorporation of unsaturated methionine analogues into proteins in vivo. J. Am. Chem. Soc. 122, 1282-1288 (2000).
3. Andruszkiewicz, R. & Rozkiewicz, D. An Improved Preparation of N2-tert-Butoxycarbonyl- and N2-Benzyloxy-carbonyl-(S)-2,4-diaminobutanoic Acids. Synth. Commun. 34, 1049-1056 (2004).
4. Szilagyi, L. & Gyorgydeak, Z. investigation of glycosyl azides and other azido sugars: Stereochemical influences on the one-bond 13C-1H coupling constants. Carbohydr. Res. 143, 21-41 (1985).
5. Macmillan, D., Danies, A. M., Bayrhuber, M. & Flitsch, S. L. Solid-Phase Synthesis of Thioether-Linked Glycopeptide Mimics for Application to Glycoprotein Semisynthesis. Org. Lett. 4, 1467-1470 (2002).
6. Davis, B. G., Lloyd, R. C. & Jones, J. B. Controlled Site-Selective Glycosylation of Proteins by a Combined Site-Directed Mutagenesis and Chemical Modification Approach. J. Org. Chem. 63, 9614-9615 (1998).
7. Chernyak, A. Y. e. a. 2-Azidoethyl glycosides: glycosides potentially useful for the preparation of neoglycoconjugates. Carbohydr. Res. 223, 303-309 (1992).
8. Fahmi, C. J. & Zhou, Z. A. Fluorogenic Probe for the Copper(I)-Catalyzed Azide-Alkyne Ligation Reaction: Modulation of the Fluorescence Emission via 3(n,p*)-1(p,p*) Inversion. J. Am. Chem. Soc. 126 (2003).
9. Lowary, T., Meldal, M., Helmboldt, A., Vasella, A. & Bock, K. Novel Type of Rigid C-Linked Glycosylacetylene—Phenylalanine Building Blocks for Combinatorial Synthesis of C-linked Glycopeptides. J. Org. Chem. 63, 9657-9668 (1998).
12. Pennica, D. e. a. Identification of Human Uromodulin as the Tamm-Horsfall Urinary Glycoprotein. Science 236, 83-88 (1987).
13. Chan, T. R., Hilgraf, R., Sharpless, K. B. & Fokin, V. V. Polytriazoles as Copper(I)-Stabilizing Ligands in Catalysis. Org. Lett. 6, 2853-2855 (2004).
14. Bradford, M. M. A rapid and sensitive method for the quantitation of microgram quantities of protein utilizing the principle of protein-dye binding. Anal. Biochem. 72, 248-254 (1976).
15. Pearce, O. M. T. et al. Glycoviruses: chemical glycosylation retargets adenoviral gene transfer. Angew. Chem. Intl Ed. 44, 1057-1061 (2005).
16. Schwarz, F. P., Puri, K. D., Bhat, R. G. & Surolia, A. Thermodynamics of Monosaccharide Binding to Concanavalin A, Pea (Pisum sativum) Lentil, and Lentil (Lens culinaris) Lectin. J. Biol. Chem. 268, 7668-7677 (1993).
17. Poretz, R. D. & Goldstein, I. J. Protein-Carbohydrate Interaction. Biochem. Pharmacol. 20, 2727-2739 (1971).
18. Lee, B. & Richards, F. M. The Interpretation of Protein Structures: Estimation of Static Accessibility. J. Mol. Biol. 55, 379-400 (1971).
19. Glocker, M. O., Borchers, C., Fiedler, W., Suckau, D. & Przybylski, M. Molecular Characterization of Surface Topology in Tertiary Structures by Amino-Acylation and Mass Spectrometric Peptide Mapping. Bioconj. Chem. 5, 583-590 (1994).
21. Santrucek, J., Strohalm, M., Kadlcik, V., Hynek, R. & Kodicek, M. Tyrosine residues modification studied by MALDI-TOF mass spectrometry. Biochem. Biophys. Res. Commun. 323, 1151-1156 (2004).
22. Aguilar, C. F. et al. Crystal structure of the beta-glycosidase from the hyperthermophilic archeon Sulfolobus solfataricus: resilience as a key factor in thermostability. J. Mol. Biol. 271, 789-802 (1997).
24. Farber, G. K. An alpha/beta-barrel full of evolutionary trouble. Curr. Opin. Struct. Biol. 3, 409-412 (1993).
25. Tamm, I. & Horsfall, F. L. A mucoprotein derived from human urine which reacts with influenza, mumps, and Newcastle desease viruses. J. Exp. Med. 95, 71-79 (1952).
26. Easton, R. L., Patankar, M. S., Clark, G. F., Morris, H. R. & Dell, A. Pregnancy-associated Changes in the Glycosylation of Tamm-Horsfall Glycoprotein. J. Biol. Chem. 275, 21928-21938 (2000).
27. van Rooijen, J. J. M., Voskamp, A. F., Kamerling, J. P. & Vliegenthart, F. G. Glycosylation sites and site-specific glycosylation in human Tamm-Horsfall glycoprotein. Glycobiology 9, 21-30 (1999).

1. A method of glycosylating a protein wherein the method comprises the steps of
i) modifying a protein to include an alkyne and/or an azide group; and
ii) reacting the modified protein in (i) with
(a) a carbohydrate moiety modified to include an azide group; and/or
(b) a carbohydrate moiety modified to include an alkyne group
in the presence of a Cu(I) catalyst.

2. A method as claimed in claim 1 wherein the modification to the protein involves the substitution of one or more amino acids in the protein with one or more unnatural amino acid analogues.

3. A method as claimed in claim 2 wherein the unnatural amino acid analogue is a methionine analogue.

4. A method as claimed in claim 3 wherein the methionine analogue is homopropargyl glycine or azido homoalanine.

5. A method as claimed in claim 1 wherein the protein comprises greater than 10 amino acids.

6. A method as claimed in claim 5 wherein the protein comprises between 10 and 1000 amino acids.

7. A method as claimed in claim 1 wherein the protein has a molecular weight greater than 10 kDa.

8. A method as claimed in claim 7 wherein the protein has a molecular weight between 10 and 100 kDa.

9. A method as claimed in claim 1 wherein the protein is selected from the group consisting of glycoproteins, blood proteins, hormones, enzymes, receptors, antibodies, interleukins and interferons.

10. A method as claimed in claim 9 wherein the protein is a hormone.

11. A method as claimed in claim 10 wherein the hormone is erythropoietin.

12. A method as claimed in claim 1, wherein the modification to the protein (step i) additionally comprises the step of modifying the protein to include a thiol group.

12. A method as claimed in claim 12 wherein the thiol group in introduced through the insertion of a cysteine residue into the amino acid sequence of the protein.

14. A method of glycosylating a protein, the method comprising the steps of
i) (a) modifying a protein to include an alkyne and/or an azide group; and
(b) before or after the modification to the protein in (a), optionally modifying a protein to include a thiol group ; and
ii) sequential reaction of the protein modified in (i) with a carbohydrate moiety (c) in the presence of a Cu (I) catalyst before or after reaction with a thiol-selective carbohydrate reagent (d)
(c) a carbohydrate moiety modified to include an azide group and/or a carbohydrate moiety modified to include an alkyne group; and
(d) a thiol-selective carbohydrate reagent.

15. A method as claimed in claim 14 wherein the thiol-selective carbohydrate reagent is a reagent which reacts with a thiol group in a protein to introduce a glycosyl residue linked to the protein via a disulfide bond.

16. A method as claimed in claim 15 wherein the thiol-selective carbohydrate reagent is a glycothiosulfonate reagent.

17. A method as claimed in claim 16 wherein the glycothiosulfonate reagent is glycomethanethiosulfonate reagent

18. A method as claimed in claim 15 wherein the thiol-selective is a glycoselenylsulfide reagents.

19. A method as claimed in claim 15 wherein the Cu(I) catalyst is selected from the group consisting of CuBr and Cul.

20. A method as claimed in claim 19 wherein the Cu(I) catalyst is Cu(I)Br.

21. A method as claimed in claim 19 wherein the Cu(I)catalyst is provided in the presence of a stabilizing amine ligand.

22. A method as claimed in claim 21 wherein the ligand is a tristriazolyl amine ligand.

23. A protein of formula (III) 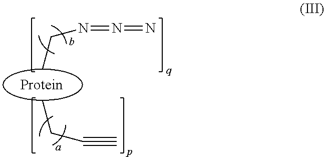
wherein a and b are integers between 0 and 5; and p and q are integers between 1 and 5.

24. A protein glycosylated by the method of claim 1.

25. A glycosylated protein of formula (IV) 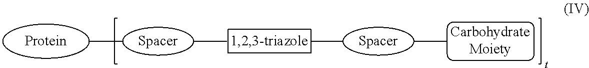
wherein t is an integer between 1 and 5; and the spacer, which may be absent, is an aliphatic moiety having from 1 to 8 C atoms.

26. A glycosylated protein as claimed in claim 25 wherein the spacer is selected from the group consisting of a C1-6 alkyl group and a C1-6 heteroalkyl.

27. A glycosylated protein as claimed in claim 26 wherein the spacer is selected from the group consisting of methyl, ethyl and CH₂(X)_{y} wherein X is O, N or S and y is 0 or 1.

28. A glycosylated protein as claimed in claim 25 wherein the protein is of formula (V) 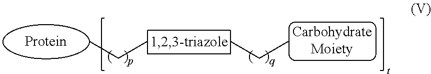
wherein p and q are integers between 0 and 5; t is an integer between 1 and 5.

29. A glycosylated protein as claimed in claim 28 wherein the protein is of formula (VI)

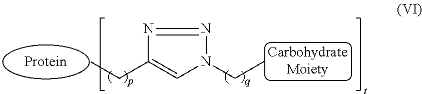

30. A glycosylated protein as claimed in claim 28 wherein the protein is of formula (VII)

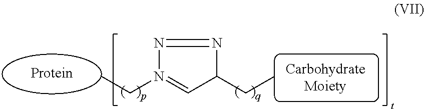

31. A glycosylated protein of formula (VIII) 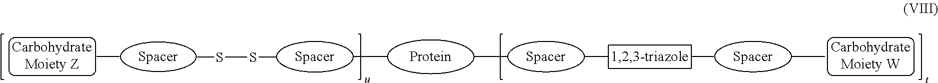
wherein u and t are integers between 1 and 5; the spacer, which may be absent, is an aliphatic moiety having from 1 to 8 C atoms; and W and Z are carbohydrate moieties that may be the same or different.

32. A glycosylated protein as claimed in claim 31 wherein the protein is of formula (IX) 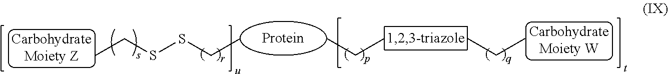
wherein p, q, r and s are integers between 0 and 5.

33. A glycosylated protein as claimed in claim 32 wherein the protein is of formula (X)

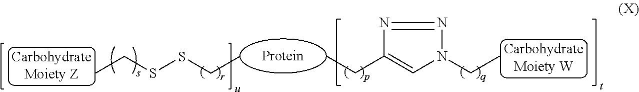

34. A glycosylated protein as claimed in claim 32 wherein the protein is of formula (XI)

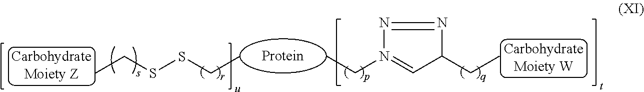

35. A protein as claimed in claim 23 wherein the protein comprises greater than 10 amino acids.

36. A protein as claimed in claim 35 wherein the protein comprises between 10 and 1000 amino acids.

37. A protein as claimed in claim 23 wherein the protein has a molecular weight greater than 10 kDa.

38. A protein as claimed in claim 37 wherein the protein has a molecular weight between 10 and 100 kDa.

39. A protein as claimed in claim 23 wherein the protein is selected from the group consisting of glycoproteins, blood proteins, hormones, enzymes, receptors, antibodies, interleukins and interferons.

40. A protein as claimed in claim 39 wherein the protein is a hormone.

41. A protein as claimed in claim 40 wherein the hormone is erythropoietin.

42. A medicament comprising a protein as claimed in in claim 23.
